(19) United States
(12) Patent Application Publication
(10) Pub. No.: US 2025/0107007 A1
(43) Pub. Date: Mar. 27, 2025

(54) FOLD FLEX CIRCUIT FOR LNOP

(71) Applicant: Masimo Corporation, Irvine, CA (US)

(72) Inventors: Benjamin C. Triman, Rancho Santa Margarita, CA (US); John Schmidt, Lake Forest, CA (US); Yassir Kamel Abdul-Hafiz, Irvine, CA (US); Ammar Al-Ali, San Juan Capistrano, CA (US)

(21) Appl. No.: 18/975,845

(22) Filed: Dec. 10, 2024

Related U.S. Application Data
(63) Continuation of application No. 17/450,912, filed on Oct. 14, 2021, now Pat. No. 12,207,419.
Application No. 17/450,912 is a continuation of application No. 16/438,267, filed on Jun. 11, 2019, now Pat. No. 11,178,776.
Application No. 16/438,267 is a continuation of application No. 15/017,505, filed on Feb. 5, 2016, now Pat. No. 10,327,337.
(60) Provisional application No. 62/112,918, filed on Feb. 6, 2015.
(60) Provisional application No. 62/212,071, filed on Aug. 31, 2015.

(51) Int. Cl. H05K 3/22; A61B 5/00; A61B 5/026; A61B 5/1455; H05K 1/18; H05K 3/00

(52) U.S. Cl. CPC H05K 3/22; A61B 5/0059; A61B 5/1455; H05K 3/0097; A61B 5/0261; A61B 2562/0233; A61B 2562/0238; A61B 2562/12; A61B 2562/164; A61B 2562/166; A61B 2562/182; H05K 1/189; H05K 3/0052; H05K 2201/0154; H05K 2201/10151; H05K 2203/1545

(57) ABSTRACT

Various sensors and methods of assembling sensors are described. In some embodiments, the sensor assembly includes a first end, a body portion, and a second end. The first end can include a neck portion and a connector portion and the second end can include a flap, a first component, a neck portion, and a second component. A method is also described for sensor folding. The method can include using a circuit with an attached emitter and a detector that is separated by a portion of the circuit. The method can also include folding the portion of the circuit such that a first fold is created through the emitter and folding the portion of the circuit such that a second fold is created such that the first fold and second fold form an angle.

### INCORPORATION BY REFERENCE TO ANY PRIORITY APPLICATIONS

This application is a continuation of U.S. patent application Ser. No. 17/450,912, filed Oct. 14, 2021, which is a continuation of U.S. patent application Ser. No. 16/438,267, filed Jun. 11, 2019, now U.S. Pat. No. 11,178,776, issued Nov. 16, 2021, which is a continuation of U.S. patent application Ser. No. 15/017,505, filed Feb. 5, 2016, now U.S. Pat. No. 10,327,337, issued Jun. 18, 2019, which claims the priority benefit under 35 U.S.C. § 119(e) of U.S. Provisional Application No. 62/112,918, filed Feb. 6, 2015, and U.S. Provisional Application No. 62/212,071, filed Aug. 31, 2015, the entire contents of which are hereby incorporated by reference and should be considered a part of this specification. Any and all applications for which a foreign or domestic priority claim is identified in the Application Data Sheet as filed with the present application are hereby incorporated by reference under 37 CFR 1.57.

### FIELD OF THE DISCLOSURE

The present disclosure relates to low-noise, disposable and reusable optical probes which may be used to sense optical energy passed through a medium to determine the characteristics of the medium.

### BACKGROUND

Energy is can be transmitted through or reflected from a medium to determine characteristics of the medium. For example, in the medical field, instead of extracting material from a patient's body for testing, light or sound energy may be caused to be incident on the patient's body and transmitted (or reflected) energy may be measured to determine information about the material through which the energy has passed. This type of non-invasive measurement is more comfortable for the patient and can be performed in real time.

Non-invasive physiological monitoring of bodily functions is often required. For example, during surgery, blood pressure and the body's available supply of oxygen, or the blood oxygen saturation, are often monitored. Measurements such as these are often performed with non-invasive techniques where assessments are made by measuring the ratio of incident to transmitted (or reflected) light through a portion of the body, for example a digit such as a finger, or an earlobe, or a forehead.

Demand has increased for disposable and reusable optical probes which are suitably constructed to provide low-noise signals to be output to a signal processor in order to determine the characteristics of the medium. Many difficulties relating to motion-induced noise have been encountered in providing such an optical probe inexpensively. A need thus exists for a low-cost, low-noise optical probe and for a method of efficient manufacturing such a probe.

### SUMMARY OF THE DISCLOSURE

The present disclosure discloses a probe for use in non-invasive optical measurements. One aspect of the present disclosure is an optical probe for non-invasive measurement of characteristics of a medium, wherein the probe has an emitter which transmits optical radiation and a detector configured to detect the optical radiation transmitted by the emitter. The probe also has a flexible circuit assembly having circuit paths for connection with the emitter and the detector

The present disclosure describes a low cost sensor and a streamlined assembly method for optimized material usage for the use of flexible printed circuit and other sensor materials. In some embodiments, the configuration of the sensors is intended to maximize the amount of material used so as to keep material cost to a minimum.

In one advantageous embodiment, the manufactured flex circuit can be folded into a number of different configurations while maintaining the properties and integrity of the original flex circuit. In this way, the same streamlined assembly method can be used to manufacture flex circuits with a plurality of configurations.

### BRIEF DESCRIPTION OF THE DRAWINGS

FIG. 1A illustrates a top view of an embodiment of a flexible circuit for use in a sensor assembly.

FIG. 1B illustrates a bottom view of an embodiment of a flexible circuit for use in a sensor assembly

FIG. 2A illustrates a top view of one embodiment of a flexible printed circuit panel array.

FIG. 2B-2C illustrate a top and bottom view of one end of the sensor assembly.

FIG. 3A illustrates an exploded view of an embodiment of a sensor assembly.

FIG. 3B illustrates one embodiment of a sheet comprising a plurality of nested top panel shields that forms a part of the flexible printed circuit panel array shown in FIG. 2.

FIG. 3C illustrates one embodiment of a sheet comprising a top panel coverlay that forms a part of the flexible printed circuit panel array shown in FIG. 2.

FIG. 3D illustrates one embodiment of a “sheet” comprising a plurality of nested traces that form a part of the flexible printed circuit panel array shown in FIG. 2.

FIG. 3E illustrates one embodiment of a sheet comprising a plurality of nested bottom coverlay that forms a part of the flexible printed circuit array shown in FIG. 2.

FIG. 3F illustrates one embodiment of a sheet comprising a plurality of nested bottom panel shields that forms a part of the flexible printed circuit array shown in FIG. 2.

FIG. 4A illustrates a top view of a first embodiment of a shield grid used in one embodiment of the sensor assembly.

FIG. 4B illustrates a top view of a second embodiment of a shield grid used in one embodiment of the sensor assembly.

FIG. 4C illustrates a top view of a third embodiment of a shield grid used in one embodiment of the sensor assembly.

FIG. 4D illustrates a top view of a fourth embodiment of a shield grid used in one embodiment of the sensor assembly.

FIG. 4E illustrates a top view of a fifth embodiment of a shield grid used in one embodiment of the sensor assembly.

FIGS. 5A-5D illustrate one embodiment of the sensor assembly.

FIG. 6A illustrates a flow chart illustrating an embodiment of a method for sensor folding.

FIG. 6B illustrates an embodiment of a sensor assembly in the method for sensor folding prior to the folding of the sensor.

FIG. 6C illustrates a first step in the method for sensor folding wherein the neck of the sensor is bent in a first direction.

FIG. 6D illustrates a second step in the method for sensor folding wherein the neck of the sensor is bent in a second direction.

### DETAILED DESCRIPTION

The present disclosure provides a low cost sensor and methods of assembly and manufacture of the low cost sensor. In some embodiments, the sensor circuits are configured such that each of the flex circuits for each of the plurality of sensors is tessellated or nested with one another as it is manufactured. In some embodiments, this configuration maximizes the number of circuits that can be manufactured and assembled from a set of materials. Such a configuration further minimizes the amount of material wasted.

The present disclosure also describes a method for assembling an L-shaped sensor or bent sensor from a straight sensor. Previous manufacturing methods for L-shaped sensors created substantial waste as the profile of the L-shaped sensor prevented the flex circuits from being printed in a staggered formation so as to maximize the use of the substrate material. By assembling the L-shaped sensor from a straight sensor, the profile of the flex circuit is minimized and the amount of waste is therefore minimized. The method of folding described below allows a plurality of different sensor shapes to be manufactured from a straight sensor.

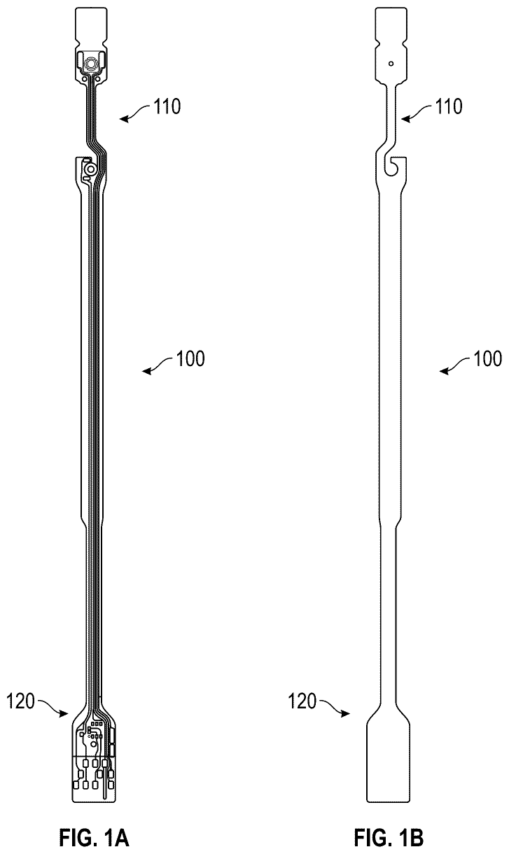

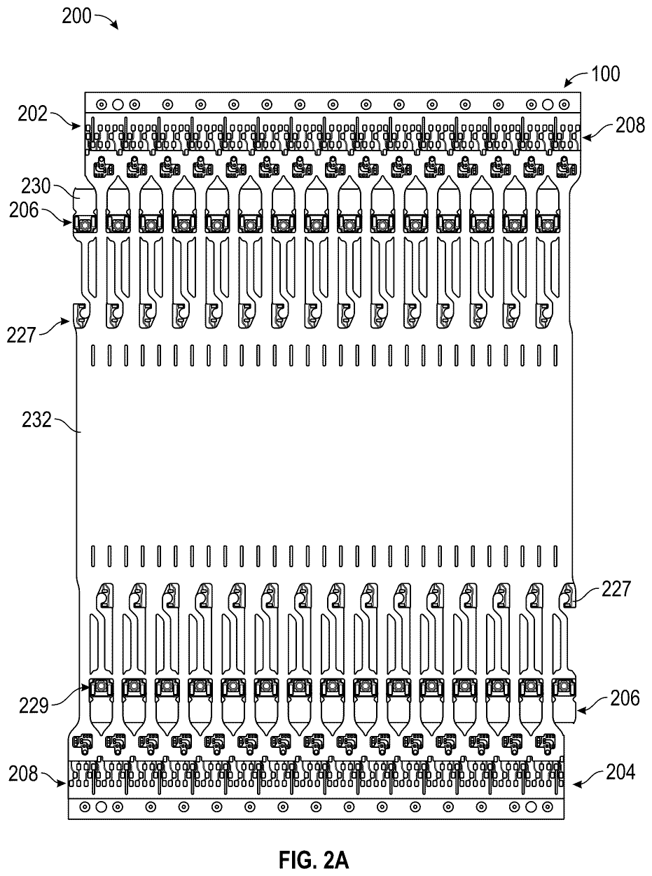

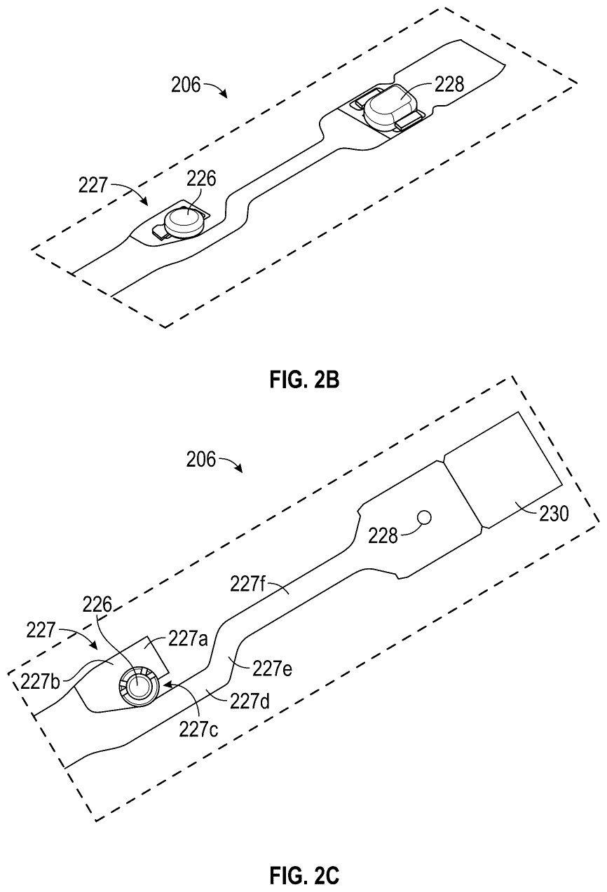

FIGS. 1-2 illustrate various views of the flex circuit 100 of the sensor assembly. FIGS. 1A-1B shows one embodiment of the flex circuit 100 of the sensor. FIG. 1A shows a top view of the flex circuit 100. The flex circuit 100 has a detector end 110 and a connector end 120. FIG. 1B shows a bottom view of the flex circuit 100 and the corresponding detector end 110 and connector end 120. As can be seen, the flex circuit 100 is generally linear and has a minimal profile that can help to maximize the number of flex circuits that can be printed on substrate material.

In some embodiments, the configuration of the flex circuit 100 can be configured to maximize the substrate material that is used and to minimize waste. FIG. 2A illustrates a flexible printed circuit panel array 200 that includes a first row of flexible circuit 202 that is nested with a second row of flexible circuits 204. In some example, the first row of flexible circuits 202 and the second row of flexible circuits 204 can be identical.

As illustrated in FIG. 2A, the first and second rows of flexible circuits 202, 204 can include a connector end 208 and a detector end 206. In some embodiments the first row of flexible circuits 202 and the second row of flexible circuits 204 are configured such that, on one end of the flexible printed circuit panel array 200, the connector end 208 of the first row of flexible circuit 202 is proximate to the detector end 206 of the second row of flexible circuit 204 and on the other end, the connector end 208 of the second row of flexible circuit 202 is proximate to the detector end 206 of the first row of flexible circuit 204.

In addition to the nested configuration, each of the flex circuits 100 has a body portion 232 that is uniform along its length which can provide for efficient machining. As illustrated in FIG. 2, the uniform body portion 232 allows for a plurality of flex circuits 100 to be aligned in a row. As well, the straight line of the body portion 232 requires a single straight-line cut to separate each flex circuit 100 from the adjacent flex circuit 100.

As noted above, the nested configuration of the first and second rows of flexible circuits significantly reduce the waste of the substrate material and increase the speed of production by generating higher yields per substrate sheet. In some examples, the percentage of raw substrate material used to form each of the flexible circuits 100 is greater than 80% and can be as high as 95% and any percentages in between. In some embodiments, the percentage of waste is as low as 5% to 20% or any percentage there between. In other examples, up to 95% of the material of the flexible printed circuit panel array 200 can be used to form each of the flex circuits 100.

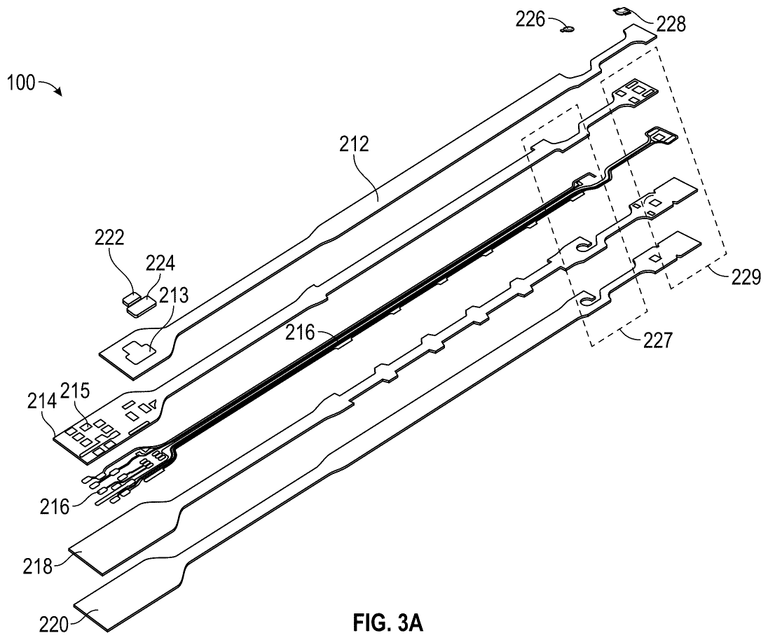

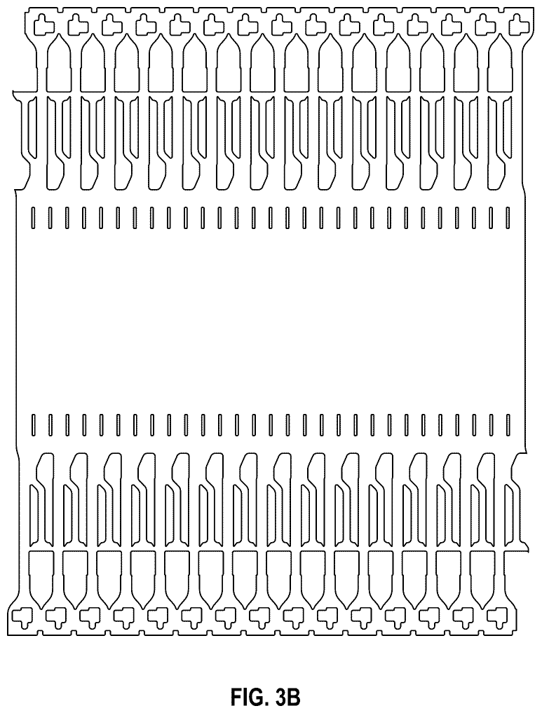

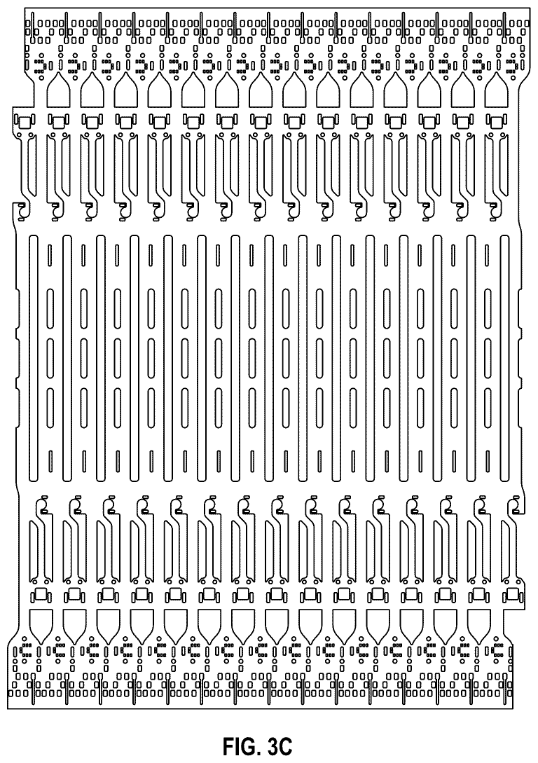

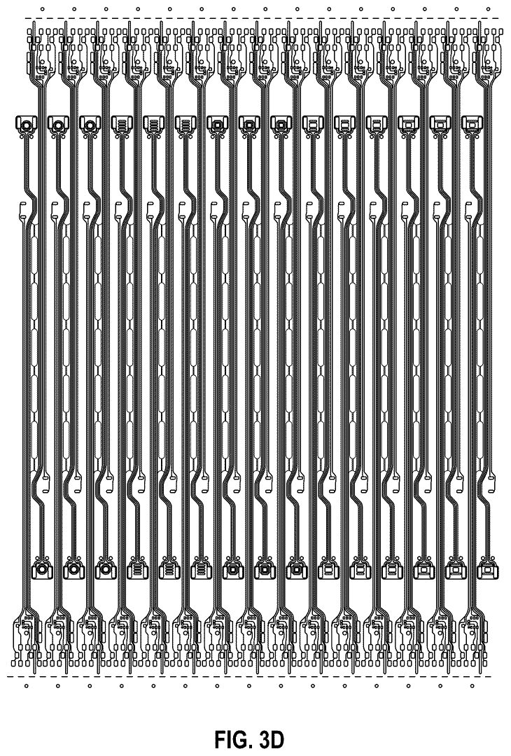

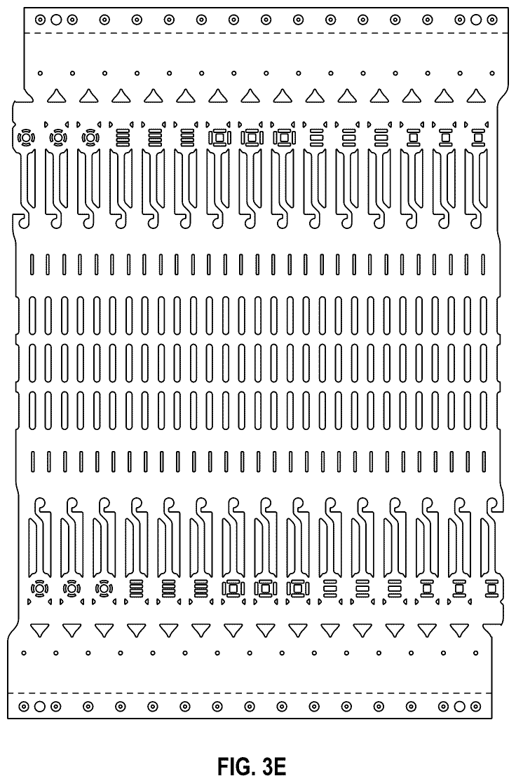

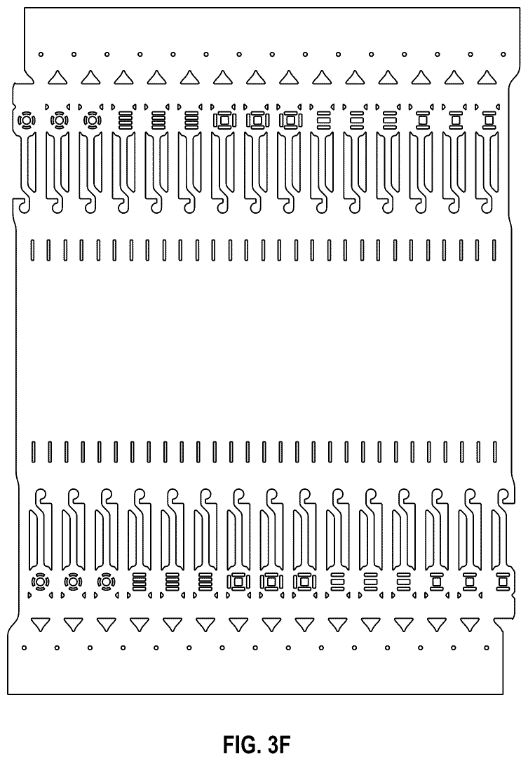

In some embodiments, each of the flex circuits 100 can be formed from a plurality of layers. FIG. 3A illustrates a perspective view of an exploded flex circuit 100 that provides a view of the construction of the flex circuit 100. In some embodiments, the flex circuit 100 includes traces 216 that are printed on a bottom coverlay 218. The traces 216 can include a copper coating while the bottom coverlay 218 can comprise a polymide material. In some examples, a top panel coverlay 214 can be layered over the bottom coverlay 218 that is printed with the traces 216. The top panel coverlay can serve as a protective layer over the 216. As will be discussed in more detail below, the top panel coverlay can include strategic openings to expose the underlying traces 216 form electrical connections on the surface of the flex circuit 100.

The flex circuit can also include a shielding layer on the top and bottom surface of the flex circuit 100 to protect the integrity of the traces 216 and to isolate the traces 216 from external factors such as radio waves, electromagnetic fields and electrostatic fields. As illustrated in FIG. 3A, the flex circuit 100 can include a top panel shield 212 that is layered over the top panel coverlay 214 and a bottom panel shield 220 that is layered under the bottom coverlay 218.

Each of the layers of the above described layers can have a nested configuration so as to form the flexible printed circuit panel array 200 illustrated in FIG. 2. For example, FIG. 3B illustrates a sheet comprising a plurality of nested top panel shields 212. FIG. 3C illustrates a sheet comprising a top panel coverlay 214. FIG. 3D illustrates a “sheet” comprising a plurality of nested traces 216. FIG. 3E illustrates a sheet comprising a plurality of nested bottom coverlay 218. Lastly, FIG. 3F illustrates a sheet comprising a plurality of nested bottom panel shields 220.

The flex circuit 100 can be configured to be attached to a plurality of components. In some examples, the flex circuit includes a resistor 222, an electrically erasable programmable read-only memory (“EEPROM”) 224, a detector 228, and an emitter 226. In some examples, the emitter 226 can be an LED.

To provide an electrical connection for the plurality of electrical components on the flex circuit 100, each of the layers of the flex circuit can include strategic openings to reveal the underlying exposed traces 217 of the traces 216. For example, the top panel coverlay 214 can include a plurality of windows 215 and the top panel shield 212 can include a window 213 to expose portions of the traces 216. The resistor 222 and EEPROM 224 can be attached to the flex circuit 100 at the window 213 to provide an electrical connection between the resistor 222 with the exposed traces 217 and an electrical connection between the EEPROM 224 and the exposed traces 217.

Similarly, as illustrated in FIGS. 2 and 3A, the flex circuit 100 can include a detector window 229 and an emitter opening 227 to accommodate a detector 228 and emitter 226 respectively. Turning first to the emitter opening 227, the flex circuit 100 can include a hooked portion to form the emitter window 227 while maintaining a reduced profile for the flex circuit 100. As can be seen in FIG. 2, the configuration of the emitter opening 227 allows each flex circuit 100 to be nested between adjacent flex circuits to form a tessellated or nested pattern. As discussed above, this can maximize the use of substrate material in the manufacturing of the flex circuit 100. The emitter 226 can be attached to the emitter opening 227 such that the emitter 226 can form an electrical connection with the hooked portion of the traces 216. As well, the hook configuration provides a circular opening that allows the light produced by the emitter 226 to be emitted.

FIG. 2B-2C illustrate an enlarged view of the detector end 206 of the flex circuit 100 with the attached detector 228 and emitter 226. FIG. 2B illustrates a top side of the detector end 206 of the flex circuit 100 and FIG. 2C illustrates a bottom side of the detector end 206 of the flex circuit 100. As discussed above, in some embodiments, the emitter opening 227 is formed from a hook configuration, the end of which is not mechanically coupled to the rest of the flex circuit 100. The hook portion of the emitter opening 227 can include a top portion 227a, a first length 227b and a second length 227d. The aforementioned three portions are configured to form an opening 227c. The top portion 227a and the first length 227b form the hook portion that the emitter 226 can attach to. In some embodiments, the second length 227d is longer than the first length 227b. As well, in some embodiments, a distance exists between the top portion 227a and the second length 227d. In some embodiments, to maintain low profile configuration of the flex circuit 100, the detector end 206 of the flex circuit includes an angled portion 227e and a length 227f that centers the detector end 206 along the length of the flex circuit 100. As is illustrated in FIG. 2C, the first length 227b and second length 227d form an opening 227c for placement of the emitter 226. The flex circuit 100 can then include an angled portion 227e that centers the detector end 206 of the flex circuit 100.

Another aspect of the configuration of the emitter opening 227 is the ability to bend one portion of the emitter opening 227. The configuration of the emitter opening 227 allows the flex circuit 227 to be bent at the second length 227d, such that a bend exists at the emitter opening 227. This can allow the straight flex circuit 100 to be bent to form a bent or L-shaped flex circuit. As will be discussed in more detail below, the hooked configuration of the emitter opening 227 provides a mechanical decoupling such that the flex circuit can be easily bent without affecting the attached emitter 226.

Turning next to the detector window 229, the detector window 229 can be formed on the surface of the top panel coverlay 214 to allow light from the light source, such as the emitter 226, to transmit through the detector window 229 and to the detector 228. In some embodiments, the detector window 229 exposes the underlying traces 216. The detector 228 can be attached to the detector window 229 such that the detector 228 forms an electrical connection with the traces 216.

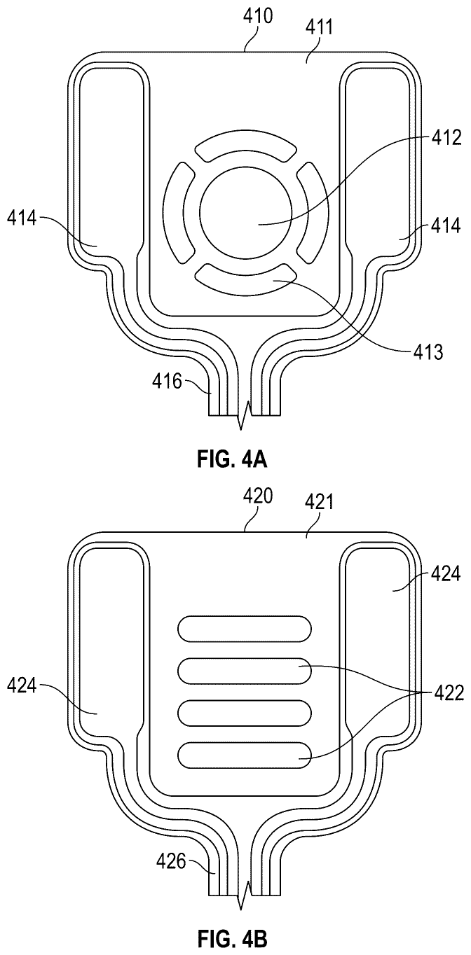

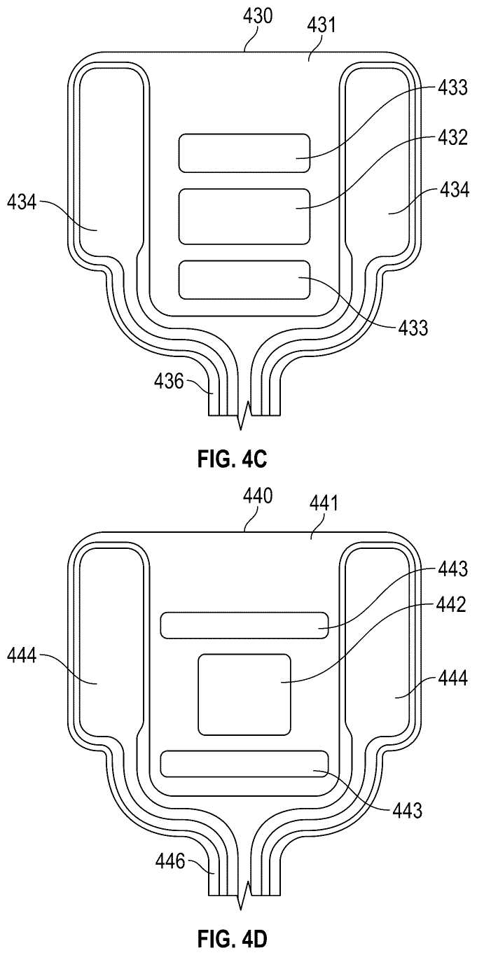

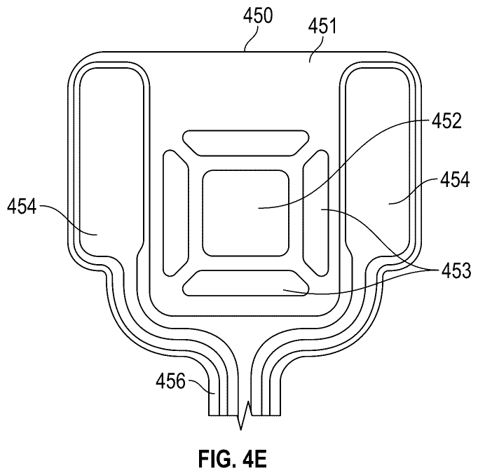

As will be discussed in FIGS. 4A-E below, the detector window 229 can vary in both shape and configuration so as to provide for varying amounts of light from the light source to enter the detector 228. The configuration and structure of each of the grid shapes can allow for the transmission of different amounts of light so as to provide a different function for the flex circuit 100.

In some embodiments, the flex circuit 100 can include a shield flap 230. In some embodiments the detector end 206 of the flex circuit 100 can form a shield flap 230. In some embodiments, the shield flap 280 can be an etched copper shield made from a copper sheet. The shield flap 230 of the detector end 206 can be configured to fold over the detector 228 to form a Faraday cage. The Faraday cage can provide additional shielding to block external electrostatic fields.

FIGS. 4A-4E illustrate an enlarged view of the various embodiments of the detector window 229. The various shield grids are designed to protect the circuits from electromagnetic noise interference while allowing as much light as possible through the grid windows. FIGS. 4A-4E illustrate the first detector window shape 410, second detector window 420, third detector window shape 430, fourth detector window shape 440, and fifth detector window shape 450 respectively. FIG. 4A illustrates the first detector window shape 410 which is located on the detector end 303 of the traces 416 layer of the flex circuit 100. The first detector window shield grid shape 410 includes a shield grid body 411 with a circular central window 412 a plurality of arc-shaped window 413, and an electrical side contact 414 on either side of the windows. In the configuration shown in the first detector window shape 410, the circular central window 412 is centered on the bottom portion of the shield grid body 411 between the pair of side contact 414. In this configuration, the first detector window shape 410 also includes four arc-shaped windows 413 that are spaced about the circular central window 412. In some embodiments, the circular central window 412 of the first detector window shape 410 allows a significant portion of light through to the detector while still blocking electromagnetic interference.

FIG. 4B illustrates the second detector window shape 420 which is located on the detector end 303 of the traces 426 layer of the flex circuit 100. The second detector window shape 420 includes a shield grid body 421 with a plurality of narrow rounded rectangular windows 422 and side contacts 424 on either side of the plurality of narrow rounded rectangular windows 422. In the configuration shown in the second detector window shape 420, the narrow rounded rectangular windows 422 have four narrow rounded rectangular windows 422 that are located on the shield grid body 421 between the two side contacts 424 on either side of the shield grid body 421.

FIG. 4C illustrates the third detector window shape 430 which is located on the detector end 303 of the traces 436 layer of the flex circuit 100. The third detector window shape 430 includes a shield grid body 431, a central window 432, a plurality of rectangular windows 433, and side contacts 434 on either side of the central windows 432. In the configuration shown in the third detector window shape 430, the plurality of rectangular windows 433 and the central window 432 are centered on the bottom portion of the shield grid body 431 between the two side contacts 434. In some embodiments, the two rectangular windows 433 are located above and below the central window 432.

FIG. 4D illustrates the fourth detector window shape 440 which is located on the detector end 303 of the traces 446 of the flex circuit 100. The fourth detector window shape 440 includes a shield grid body 441, a central window 442, a plurality of narrow rectangular windows 443, and a side contact 444 on either side of the central window 442. In the configuration shown in the fourth detector window shape 440, the narrow rectangular window 443 and the central window 442 are centered on the bottom portion of the shield grid body 441 between the two side contacts 444. In some embodiments, the two narrow rectangular window 443 are located above and below the central window 442.

Lastly, FIG. 4E illustrates the fifth detector window shape 450 which is located on the detector end 303 of the traces 456 of the flex circuit 100. The fifth detector window shape 450 includes a shield grid body 451, a central window 452, a plurality of side contact 454, and a side contact 454 on either side of the central window 452. In the configuration shown in the fifth detector window shape 450, the central window 452 and the plurality of trapezoidal window 453 are located on the bottom portion of the shield grid body 451. In some embodiments, each of the plurality of trapezoidal window 453 is located one side of the central window 452 such that the shorter end of the trapezoid is proximate to a side of the central window 452.

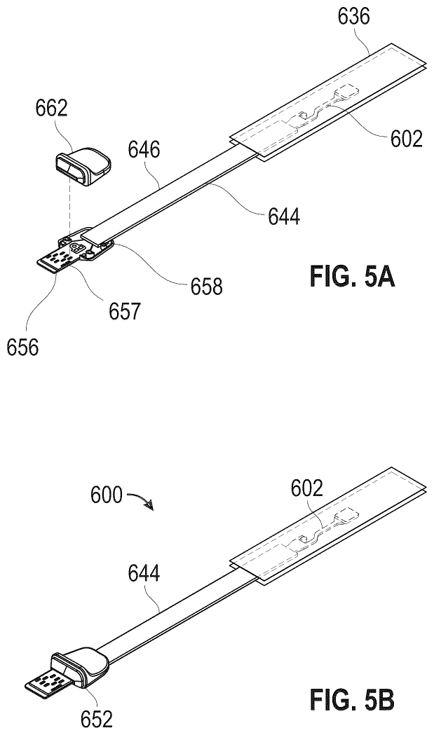

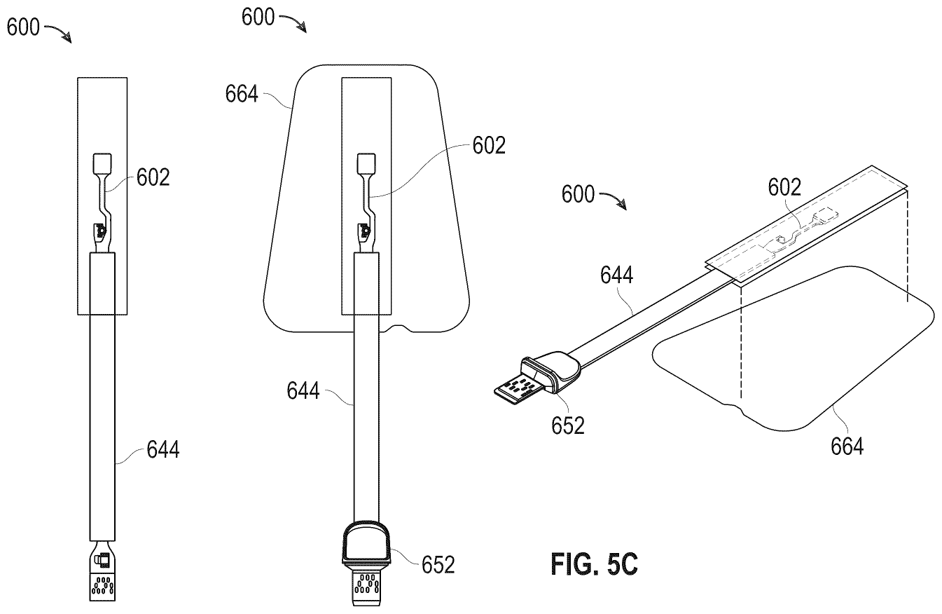

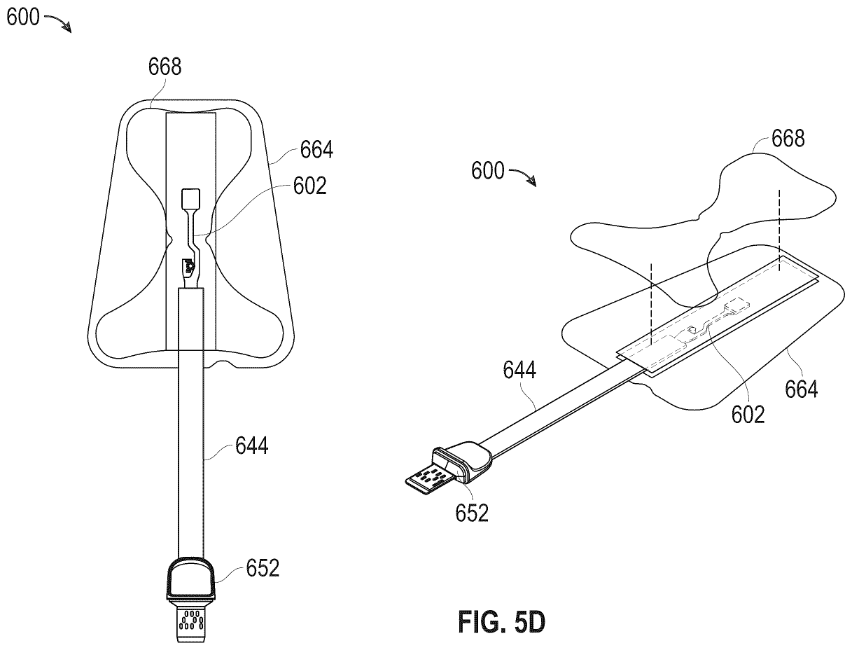

The configuration of the two sheet flexible printed circuit panel array 300 provides for a larger number of sensors to be assembled at the same time. Once all of the components have been attached and assembled on each of the sensor assemblies, each of the sensor assemblies 644 can be sealed in protective material. As illustrated in FIGS. 5A-5D, in some embodiments, the sensor assemblies can include top and bottom portions 646. For example, in some embodiments the sensor assemblies can covered on both top and bottom with a layer of foam 646. The foam covering covers the flex circuit and traces and forms a cable covering which extends from the emitter and detector assemblies to a connector end of the flex circuit. In some embodiments, a top foam 646 and a bottom foam 646 can be sealed together to sandwich the flex circuit such that the sensor assembly is entirely covered by the foam.

In some embodiments, each of the sensor assemblies 644 can include a top head tape 636 and a bottom head tape 636 attached to cover each individual sensor. In some embodiments, the top head tape 636 can be the same size as the bottom head tape 636. In some embodiments the top head tape 636 can have a design such as sensor artwork or logos printed on its top surface. In some embodiments, after the bottom head tape 636 and the top head tape 636 have been attached to the sensor assembly, the sensor assembly can be laminated.

Each of the sensor assemblies can further include a bottom and top connector tab. The connector tab provides the sensor assembly 644 with a structure to allow the sensor 644 to attach to a connector. FIG. 5A illustrates an example of a sensor assembly 644 with connector tabs attached. In some embodiments, the bottom connector stiffener 656 can include a flex circuit mating area 658. In some embodiments, the resistor end of the sensor 644 is placed such that the exposed traces discussed in FIG. 2 lie on the surface of the proximal tongue 657. The flex circuit mating area 658 can be configured to connect with the top portion of the connector assembly. Prior to the placement of the sensor 644 on the bottom connector stiffener 656, a bonding agent such as glue or epoxy can be applied to the bottom connector stiffener 656. Once applied, the sensor 644 can placed on the bottom connector stiffener 656 with component side facing upwards. In the embodiment pictured in FIG. 5A, the flex circuit mating area 658 portion of the bottom connector stiffener 656 is located on either side of the sensor 644. Once the sensor 644 is attached to the bottom connector stiffener 656, the top portion of the connector tab is attached to secure the sensor 644. In some embodiments, the underside of the top connector stiffener 662 has a mating area that corresponds to the flex circuit mating area 658 such that the top connector stiffener 662 and flex circuit mating area 658 are secured together. In some embodiments, the top connector stiffener 662 and flex circuit mating area 658 are secured using a locking mechanism or a fastener. The top connector stiffener 662 and the bottom connector stiffener 656 can be secured together by a press fit, interference fit, a snap fit, etc. FIG. 5B illustrates the sensor 644 with the connector stiffener 652 assembled onto the connector end of the sensor 644.

Finally, the sensor assembly 644 can optionally include a printed liner and applicator tape. FIGS. 5C-5D illustrate a top perspective view of the sensor 644 with the added printed liner and the applicator tape. FIG. 5C provides a top view of the sensor 644 and a top and perspective view of the sensor 644 with the printed liner 664 added. The printed liner 664 can be printed with a variety of designs and/or colors. As can be seen in FIG. 5C, the printed liner 664 can be long enough to fit the length of the head tape 636 section of the sensor assembly 644. FIG. 5D illustrates a top and perspective view of the sensor assembly 644 with printed liner 664 and added applicator tape 668. The applicator tape 668 can have a variety of shapes and sizes. In some embodiments, the 688 has a length and width that can fit onto the printed liner 664.

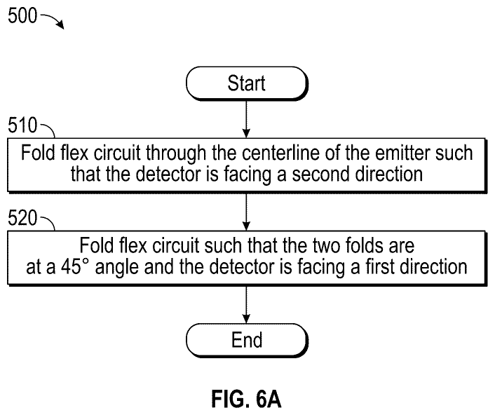

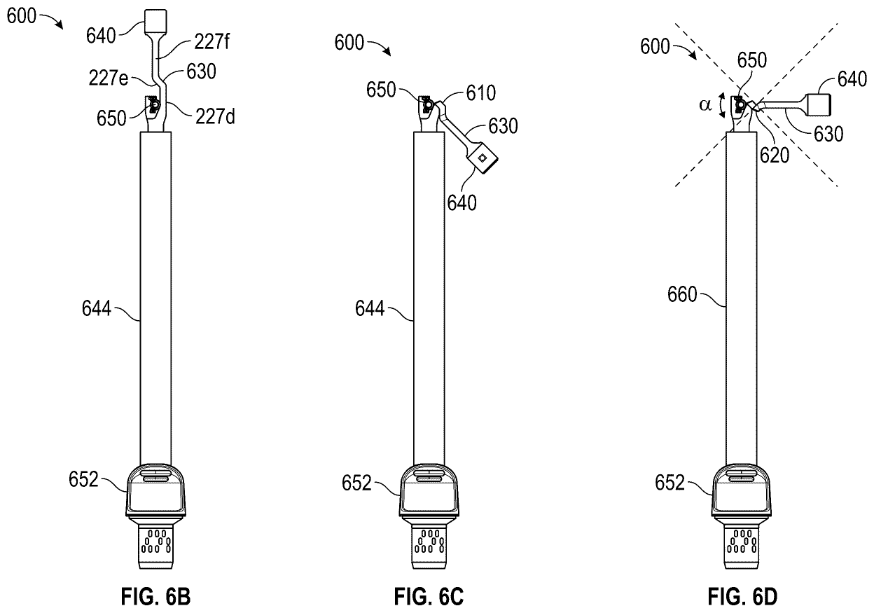

As described above, another benefit of the present configuration of the flex circuit design is the ability to assemble a bent sensor from the linear flex circuit described above. FIG. 6A illustrates a flowchart that describes an embodiment of a method of sensor folding 500. FIGS. 6B-6D illustrate a method of sensor folding 800 that corresponds with the steps of the flowchart shown in FIG. 6. As discussed earlier, although FIGS. 6A-D describe the formation of an “L-shaped” sensor, the steps described can be applied to fold the flex circuit into a sensor that is bent at an angle greater or less than 90 degrees.

The method of sensor folding 500 can include block 510 which describes folding the flex circuit through the centerline of the emitter such that the detector is facing a second direction. FIG. 6B illustrates the sensor prior to folding. As seen in previous figures, the sensor 644 includes a detector 640, an emitter 650, and a neck 630 connecting the detector 640 with the emitter 650. As discussed above, the neck 630 is formed from a second length 227d, an angled portion 227e and another length 227f which is configured to maintain the straight configuration of the flex circuit 100. As discussed above, the second length 227d is initially angled to one side to form the opening 227c that accommodates the emitter 226.

In the configuration of FIG. 6B, the detector 640 and emitter 650 both face a first direction such that the detector window and emitter opening both face a second direction. FIG. 6C illustrates the sensor 644 with a first fold 610 through the centerline of the emitter 650. In this embodiment, the first fold 610 is at a 45 degree angle with the remaining length of the sensor 644. In other embodiments, the angle of the first fold 610 can range from 0-180 degrees. The first fold 610 creates a fold in the neck emitter opening such that the detector 640 is facing a second direction, with the detector window facing a first direction.

Once the first fold is made, the method of sensor folding 500 can further include block 520 which describes folding the flex circuit a second time such that the two folds—the first fold and the second fold—form a 45 degree angle and the detector is now facing a first direction. FIG. 6D illustrates the second fold 620 of the L-shaped sensor 660. The second fold 620 and the first fold 610 form fold angle α. In some embodiments, the fold angle α is at a 45 degree angle. The second fold 620 also turns the detector 640 such that it is facing a first direction and the detector window is facing a second direction. In this way, the direction of the detector 640 and detector window are facing the same directions as they were prior to folding. After folding, a head tape, applicator tape and liner can be added to finish the sensor similar to those described above. Moreover, the folding of the sensor flex circuit can occur at any time during the manufacturing process and is not limited to any particular sequence of sensor construction.

Finally, all of the sensors discussed above can be reprocessed or refurbished. The reprocessing or refurbishing of physicological sensors involves reusing large portions of an existing sensor. The reprocessed or refurbished sensor therefore has material costs that are significantly lower than making an entirely new sensor. In one example, the reprocessing or refurbishing of the sensor can be accomplished by replacing the adhesive portion of the sensor and reusing the sensing components. In other examples, the process for reprocessing or refurbishing sensors involves replacing the sensing components of the sensor. One such example is described in U.S. Pat. No. 8,584,345 entitled “Reprocessing of a physiological sensor,” which is assigned to Masimo Corporation, Irvine, California, and incorporated by reference herein.

Although this disclosure has been disclosed in the context of certain preferred embodiments and examples, it will be understood by those skilled in the art that the present disclosure extends beyond the specifically disclosed embodiments to other alternative embodiments and/or uses of the disclosure and obvious modifications and equivalents thereof. In addition, while a number of variations of the disclosure have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. Accordingly, it should be understood that various features and aspects of the disclosed embodiments can be combined with or substituted for one another in order to form varying modes of the disclosed

What is claimed is:

1. An optical sensor assembly comprising:
a first end;
a second end; and
a body portion extending between the first end and the second end;
the first end comprising a connector;
the second end comprising:
a flap,
a component comprising a detector,
a neck portion, and
a hooked portion forming an emitter window, the hooked portion being adapted for attachment of an emitter.

2. The sensor assembly of claim 1, further comprising the emitter attached to the hooked portion.

3. The sensor assembly of claim 1, wherein the flap is configured to fold over the component.

4. The sensor assembly of claim 1, further comprising a plurality of windows configured to allow light from the emitter to transmit through the plurality of windows to the component, wherein the component is configured to attach to the plurality of windows.

5. The sensor assembly of claim 4, wherein the plurality of windows includes a circular window and a plurality of arc-shaped windows.

6. The sensor assembly of claim 5, wherein the plurality of arc-shaped windows are disposed about the circular window.

7. The sensor assembly of claim 5, wherein the plurality of arc-shaped windows includes four arc-shaped windows.

8. The sensor assembly of claim 4, wherein the structure includes a plurality of rectangular windows.

9. The sensor assembly of claim 8, wherein the plurality of rectangular windows comprises four rectangular windows between sides contacts on either side of the plurality of rectangular windows.

10. The sensor assembly of claim 8, wherein the plurality of rectangular windows comprises a central wider window and narrower rectangular windows above and below the central wider rectangular window.

11. The sensor assembly of claim 8, wherein the plurality of rectangular windows comprises a central four-sided window and a narrower rectangular window along each side of the central four-sided window.

12. The sensor assembly of claim 1, wherein the hooked portion is formed from the neck portion.

13. The sensor assembly of claim 12, wherein a curved portion is attached to the neck portion.

14. The sensor assembly of claim 12, wherein the neck portion is curved.

15. The sensor assembly of claim 1, wherein the sensor assembly is configured to fold such that a first fold is created to form a first angle.

16. The sensor assembly of claim 15, wherein the sensor assembly is configured to fold such that a second fold is created, wherein the first fold and the second fold form a second angle.

17. The sensor assembly of claim 16, wherein the first or second angle is a 45 degree angle.

18. The sensor assembly of claim 1, wherein the connector comprises a top stiffener and a bottom stiffener secured together by a press fit, an interference fit, or a snap fit.

19. The sensor assembly of claim 18, wherein the top stiffener and the bottom stiffener include corresponding flex circuit mating areas.
